(19) United States
(12) Patent Application Publication
(10) Pub. No.: US 2006/0100263 A1
(43) Pub. Date: May 11, 2006

(54) Antipyretic compositions and methods

(76) Inventors: Anthony Basile, Hoboken, NJ (US); Warren Stern, Plymouth, MA (US)

Correspondence Address: Jeffrey J. King;GRAYBEAL JACKSON HALEY LLP, Suite 350, 155 - 108th Avenue NE, Bellevue, WA, 98004-5901, US

(21) Appl. No.: 11/263,045

(22) Filed: Oct. 31, 2005

Related U.S. Application Data
(60) Provisional application No. 60/625,207, filed on Nov. 5, 2004.

(51) Int. Cl. A61K 31/403

(52) U.S. Cl. 514/412

(57) ABSTRACT

Methods and compositions containing bicifadine are provided for the treatment and prevention of hyperthermia in mammalian subjects. The methods and compositions may be used to prevent or treat fever, pyresis, menopausal hot flashes; peri menopausal hot flashes, postmenopausal hot flashes, hot flashes caused by anti-estrogen therapy, hot flashes secondary to surgical removal of estrogen producing tissue, hot flashes caused by radiation therapy, malignant hyperthermia, serotonin syndrome, heat stroke, febrile seizures, and neuroleptic malignant syndrome, among other conditions. Additional compositions and methods are provided employing bicifadine to treat pyresis and simultaneously elicit an analgesic response in mammalian subjects. Yet additional compositions and methods are provided which employ bicifadine in combination with a second antipyretic agent, a second analgesic agent, or a different therapeutic agent to yield more effective antipyretic treatment tools, and/or dual activity therapeutic methods and formulations useful to prevent or reduce hyperthermia and one or more additional symptoms (e.g., pain, or depression) in mammalian subjects.

### RELATED APPLICATIONS

This application claims priority to U.S. Provisional patent application Ser. No. 60/625,207, filed Nov. 5, 2004.

### TECHNICAL FIELD

The present invention relates to methods and compositions for controlling body temperature changes in mammalian subjects. More specifically, the invention relates to methods and compositions for preventing and/or treating hyperthermia and related conditions in mammals.

### BACKGROUND

Thermal homeostasis in mammals is maintained through a complex interplay between regulatory neurons in the central and autonomic nervous systems. In humans, normal body temperature is tightly controlled between 97.6° F. and 98.5° F. (Mackowiak, 1997), primarily by neurons in the preoptic nucleus of the anterior hypothalamus (POAH) and the septal nuclei. Neurons of the POAH process thermal signals generated in the body core and periphery (skin) to maintain normal body temperature by evoking physiological and/or behavioral responses that regulate heat production or dissipation, including physical activity, sweating, shivering, vasodilation or vasoconstriction, mobilization of energy stores through thyroid hormone and glucagon release, and metabolic thermogenesis from white and brown fat.

These homeostatic mechanisms are typically invoked through activation of the sympathetic (peripheral noradrenergic) nervous system. Released norepinephrine can then activate α₁ adrenergic receptors in the vasculature to mediate vasoconstriction and heat retention (Frank et al., 1997), while β₃ receptor activation invokes thermogenesis (i.e., production of heat from white and brown fat stores) (Granneman et al., 2003, Cannon and Nedergaard, 2004). Thermogenesis and heat retention can also be activated by cholinergic mechanisms (e.g., to elicit shivering), and/or serotonergic mechanisms (e.g., activation of vascular 5-HT₂A receptors to cause vasoconstriction) (Blessing and Seaman, 2003).

Failure of the body to maintain normal thermal homeostasis results in either hypothermia (lowering of body temperature below normal), or hyperthermia (elevation of body temperature above normal), either of which can lead to a wide range of adverse sequelae, including organ failure and even death.

The most common manifestation of hyperthermia is pyresis, or fever. Fever may be intermittent, characterized by daily spikes followed by a return to normal temperature, or remittent, in which the temperature does not return to normal. Fever may be caused by infection (e.g., viral, bacterial or fungal infection) or mediated by various noninfectious conditions (e.g., inflammatory, neoplastic, or immunological disorders), and may result from elevated heat production and/or increased heat conservation (e.g., reduced heat dissipation capacity).

Fevers caused by infection typically result from a release of exogenous pyrogens by infectious agents, for example bacterial lipopolysaccharides. These exogenous pyrogens trigger endogenous pyrogens such as IL-1β, IL-6, tumor necrosis factor, the interferons, prostaglandains and the gp 130 receptor-activating family (interleukin-6, interleukin-11, leukemia inhibitory factor, ciliary neurotropic factor, and oncostatin M) which initiate metabolic changes in the hypothalamic thermoregulatory center.

Non-infectious causes of hyperthermia may include thermoregulatory disorders, for example: serotonin syndrome; neuroleptic malignant syndrome; malignant hyperthermia; central nervous system thermoregulation disorders; impairment of heat loss, and/or thermal challenge (as typically observed in heat stroke); endocrine disorders such as hyperthyroidism, pheochromocytoma and menopause (including peri menopausal and post menopausal thermal disorders); and severe illnesses, such as stroke and cancer.

Hyperthermia may also be induced by drugs such as serotonergics (e.g., MDMA intoxication; Nisijima et al., 2001, Sprague et al., 2003, Birmes, 2003), antipsychotics (e.g., haloperidol, which may trigger neuroleptic malignant syndrome; Fricchione et al., 2000, Bhanushali and Tuite, 2004), anticholinesterases, sympathomimetics, (Halloran and Bernard, 2004), and halothane in subjects with mutations of the ryanodine receptor.

If uncontrolled, all types of hyperthermia can lead to systemic damage, including central nervous system (CNS) damage, organ failure, and even death.

In most cases, hyperthermia is considered a serious condition by medical practitioners and aggressive efforts are undertaken to reduce pyresis if it extends significantly beyond the normal temperature range and/or for an extended period of time. Attempts to reduce hyperthermia are undertaken with the objectives of increasing the comfort of patients and, more importantly, to avoid morbidity, including systemic damage such as organ injury or failure, resulting from chronic elevations of body temperature (Aronoff and Neilson, 2001, Greisman and Mackowiak, 2002). Common sequelae to hyperthermia which are secondary or attendant clinical targets for intervention can include febrile seizures, myocardial infarction, rhabdomyolysis, pancreatitis, hepatic failure, respiratory distress, ataxia, cognitive impairment, myoglobinuria, renal failure, liver damage and disseminated intravascular coagulopathy, among other adverse, hyperthermia-associated conditions (see, e.g., Greisman and Mackowiak, 2002, Lazarus et al., 1989).

Current therapies for hyperthermia include the use of conventional antipyretic drugs such as acetaminophen, non-steroidal anti-inflammatories (NSAIDS), anti-inflammatories such as glucocorticoids, and hormone replacement therapy in the treatment of endocrine disorders. However, these therapies all have side effects and are not necessarily effective for all types of hyperthermia. For example, NSAIDS are associated with a significant risk of gastrointestinal and renal toxicities, as well as a potential for causing Reye's syndrome. Glucocorticoids are also powerful immunosuppressants, which may leave patients susceptible to infection. Prazosin and other alpha-adrenergic antagonists may cause hypotension, which can lead to tachycardia and syncope. Hormone replacement therapy, such as estrogen replacement therapy, is contraindicated in many patients, particularly those with a history of cancer or venous thromboembolism. Hormone replacement therapy has also been linked to increased risks of cancer, coronary artery disease, and stroke.

In view of the foregoing, there remains an important, unmet need in the art for alternative compositions and methods to prevent and/or reduce hyperthermia and associated conditions in mammalian subjects. A related need exists for improved tools and methods for prophylaxis and treatment of specific forms of hyperthermia, for example to treat peri- and post menopausal hot flashes, and to ameliorate other forms of intermittent, recurring hyperthermia.

### SUMMARY OF THE DISCLOSURE

It is therefore an object of the present invention to provide novel and improved compositions and methods for treating and managing hyperthermia in mammalian subjects, including humans.

It is a further object of this invention to provide compositions and methods for preventing hyperthermia and/or reducing elevated body temperature and attendant adverse conditions in mammalian subjects, particularly those suffering from impaired thermal homeostasis.

The invention achieves these objects and satisfies additional objects and advantages by providing methods and compositions for treating and/or preventing hyperthermia in mammalian subjects, using the non-narcotic analgesic, bicifadine.

Useful bicifadine compounds within the formulations and methods of the invention include compounds in the class of 1-phenyl-3-azabicyclo[3.1.0]hexanes, particularly such compounds having at least one substituent on the phenyl ring. Useful forms of bicifadine for use herein include various pharmaceutically acceptable salts, polymorphs, solvates, hydrates, and/or prodrugs of bicifadine, or combinations thereof. In exemplary embodiments, the compositions and methods of the invention may employ a bicifadine HCl compound as an active antipyretic agent.

Mammalian subjects amenable for treatment according to the methods of the invention include, but are not limited to, subjects presenting with clinical grade fevers, or pyresis; endocrine disorders; menopausal hot flashes; peri menopausal hot flashes; post menopausal hot flashes; hot flashes caused by anti-estrogen therapy; hot flashes secondary to surgical removal of estrogen producing tissue; hot flashes caused by radiation therapy; malignant hyperthermia; serotonin syndrome; heat stroke; febrile seizures; and neuroleptic malignant syndrome.

These and other subjects are effectively treated, prophylactically and/or therapeutically, by administering to the subject an antipyretic effective amount of bicifadine sufficient to prevent or reduce temperature elevation, or other hyperthermic response(s) or associated condition(s), in the subject, and/or to prevent or reduce one or more symptom(s) secondary or attendant to hyperthermia in the subject. As noted above, in various embodiments of the invention the antipyretic methods and formulations of the invention may employ bicifadine in a variety of forms, including its pharmaceutically acceptable salts, polymorphs, solvates, hydrates, prodrugs, and/or combinations thereof, including an exemplary form of bicifadine, bicifadine HCl, as used in the examples herein for illustrative purposes.

Within additional aspects of the invention, combinatorial formulations and methods are provided which employ an effective amount of bicifadine and one or more additional active agent(s) that is/are combinatorially formulated or coordinately administered with bicifadine to yield an antipyretic composition or coordinate treatment response. Exemplary combinatorial formulations and coordinate treatment methods in this context employ bicifadine in combination with one or more additional antipyretic agents, and/or in combination with one or more additional analgesic agents.

### BRIEF DESCRIPTION OF THE DRAWING

FIG. 1 is a graph depicting antipyretic activity of orally-administered bicifadine HCl in rats in which fever was induced by subcutaneous administration of 10 ml/kg of 40% brewers' yeast 18 hours prior to bicifadine administration.

### DETAILED DESCRIPTION OF EXEMPLARY EMBODIMENTS

The instant invention provides novel methods and compositions for preventing and/or treating hyperthermia, fever, or pyresis in mammalian subjects. In various embodiments, the methods and compositions are effective for preventing elevation of body temperature above a normal body temperature range, and/or for lowering body temperature that has elevated above normal body temperature range in mammalian subjects suffering from impairment of thermal homeostasis.

Antipyretic formulations and methods provided herein employ bicifadine as a novel antipyretic effective agent. Within these formulations and methods, the bicifadine may be provided in any of a variety of forms, including any pharmaceutically acceptable salt, solvate, polymorph or prodrug of bicifadine, and/or combinations thereof. In exemplary compositions and methods, bicifadine HCl is effectively used to treat hyperthermia in mammalian subjects.

A broad range of mammalian subjects, including human subjects, are amenable for treatment using the formulations and methods of the invention. These subjects include, but are not limited to, human and other mammalian subjects presenting with clinical grade fever, or intermittent, acute, or chronic hyperthermia associated with infection, inflammation, injury, endocrine disorders, menopausal hot flashes, peri menopausal hot flashes, post menopausal hot flashes, hot flashes caused by anti-estrogen therapy, hot flashes secondary to surgical removal of estrogen producing tissue, hot flashes caused by radiation therapy, malignant hyperthermia, serotonin syndrome, heat stroke, febrile seizures, and neuroleptic malignant syndrome, among various other disorders that are triggered by or associated with impaired thermal homeostasis.

Within the methods and compositions of the invention, bicifadine is effectively formulated or administered as an antipyretic agent effective for treating hyperthermia and/or related disorders of thermoregulation in mammals. In exemplary embodiments, bicifadine HCl is shown to be an effective antipyretic agent in pharmaceutical formulations and methods. It is further apparent from the present disclosure that additional, pharmaceutically acceptable bicifadine compounds, complexes, derivatives, salts, solvates, polymorphs, and prodrugs, and combinations thereof will be comparably effective antipyretic agents within the methods and compositions of the invention.

Antipyretic compositions, including pharmaceutical formulations, of the invention comprise an antipyretic effective amount of a bicifadine, which is antipyretically effective for prophylaxis and/or treatment of hyperthermia (and/or disorders of thermoregulation, and/or various associated or attendant symptoms of hyperthermia) in mammalian subjects. Within exemplary embodiments these compositions are effective within in vivo treatment methods to alleviate pyresis associated with inflammation, infection, injury, post surgical reaction, menopause (including menopausal, peri menopausal, and post menopausal hotflashes), surgical removal of estrogen producing tissue, radiation therapy, heat stroke, serotonin syndrome, neuroleptic malignant syndrome, malignant hyperthermia, stroke, and/or cancer.

Antipyretic compositions of the invention typically comprise an antipyretically effective amount or unit dosage of bicifadine, which may be formulated with a pharmaceutically acceptable carrier and/or various excipients, vehicles, stabilizers, buffers, etc. Antipyretic effective amounts of bicifadine (e.g., a unit dose or concentration of bicifadine HCL, or of any selected pharmaceutically acceptable salt(s), solvate(s), polymorph(s) and/or prodrug(s) of bicifadine) will be readily by those of ordinary skill in the art, depending on clinical and patient-specific factors. Suitable effective unit dosage amounts for mammalian subjects, including humans may range from 25 to 1800 mg, 50 to 1000 mg, 75 to 900 mg, 100 to 750 mg, or 150 to 500 mg. In certain embodiments, the antipyretic effective dosage of bicifadine may be selected within narrower ranges of, for example, 10 to 25 mg, 30-50 mg, 75 to 100 mg, 100 to 250 mg, or 250 to 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In one exemplary embodiment, dosages of 100 to 600 mg are administered two to three times per day. In another embodiment dosages of 100 to 400 mg are administered twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing and mode of delivery of compositions of the invention comprising an effective amount of bicifadine will routinely be adjusted on an individual basis depending on such factors as weight, age, gender, and condition of the individual, the acuteness of the hyperthermia and/or related symptoms, whether the administration is prophylactic or therapeutic, and on the basis of other factors known to effect drug absorption and thermal homeostasis.

An effective dose or multi-dose treatment regimen for the instant antipyretic formulations will be ordinarily be selected to approximate a minimal dosing regimen that is necessary and sufficient to substantially prevent or alleviate a state of hyperthermia in the subject, and/or to substantially prevent or alleviate one or more symptoms associated with an impairment of thermal homeostasis in the subject. In the case of fever associated with infection, for example, a dosage and administration protocol will often include repeated dosing therapy over a course of several days, or even one or more weeks. In the case of menopausal or peri menopausal hot flashes, an effective treatment regimen may involve prophylactic dosage administered on a daily or multi-dose/day basis lasting over a course of days, weeks, months, or even years. In contrast, patients suffering from acute hyperthermia, such as drug-induced hyperthermia (e.g., as elicited by MDMA overdose) may require rapid intervention with antipyretic bicifadine formulations and methods, such as by intravenous drip or infusion. In these and clinically related cases, the effective dose of bicifadine may comprise, for example, 70 to 800 mg per day, given in multiple IV injections of 25 to 100 mg, or an infusion of 0.5 to 1 mg/min, administered in a volume of 0.5 to 1 ml.

The therapeutic effectiveness of antipyretic bicifadine treatment according to the invention may be demonstrated, for example, by a decrease in elevated body temperature of a hyperthermic subject, of from about 0.5 to as much as 6° F. or more. In exemplary embodiments, the compositions and methods of the invention are antipyretically effective to bring about a decrease in elevated body temperature in hyperthermic subjects of from at least about 0.5 to 1.5° F., often from about 1 to 4° F., and as much as from about 3 to 7° F. Antipyretic effectiveness may also be demonstrated in certain subjects by a decrease in any one or assemblage of symptoms caused by, or associated with, pyresis or impairment of thermal homeostasis in mammalian subjects. For each of the indicated conditions described herein, test subjects will exhibit a 10%, 20%, 30%, 50% or greater reduction, up to a 75-90%, or 95% or greater, reduction, in one or more symptoms caused by, or associated with, pyresis or impairment of thermal homeostasis in the subject, compared to placebo-treated or other suitable control subjects.

Within other exemplary embodiments of the invention, bicifadine formulations and methods are provided for effective management, prophylaxis, and/or treatment of serotonin syndrome. Serotonin syndrome is caused by excess stimulation of post-synaptic 5-hydroxytryptamine receptors in the brain stem and spinal cord. Adverse symptoms associated with this condition include euphoria, drowsiness, sustained rapid eye movement, overreaction of the reflexes, rapid muscle contraction and relaxation in the ankle causing abnormal movements of the foot, clumsiness, restlessness, feeling drunk and dizzy, muscle contraction and relaxation in the jaw, sweating, intoxication, muscle twitching, rigidity, high body temperature, mental status, shivering, diarrhea, loss of consciousness and death. Serotonin syndrome is generally induced by a combination of two or more drugs, one of which is often a selective serotonergic medication. The drugs which are known to frequently contribute to this condition are combinations of monoamine oxidase inhibitors (MAOIs) with fluoxetine (Prozac) and other selective Serotonin Reuptake Inhibitors (SSRIs) or other drugs that have a powerful effect upon serotonin such as clomipramine (Anafranil), or trazadone (Deseryl). Acute serotonin syndrome can also be caused by an overdose of MDMA (Ecstasy).

The methods and formulations of the invention for treating serotonin syndrome employ an effective amount of bicifadine in a pharmaceutical composition suitable for administration to mammalian subjects. The methods and formulations deliver an effective amount of bicifadine to prevent, or substantially alleviate, one or more of the above-identified adverse symptoms associated with serotonin syndrome. Thus, following administration of the inventive bicifadine formulation or method, test subjects will exhibit a 10%, 20%, 30%, 50% or greater reduction, up to a 75-90%, or 95% or greater, reduction, in one or more symptoms associated with serotonin syndrome, compared to placebo-treated or other suitable control subjects.

Within additional exemplary embodiments of the invention, bicifadine formulations and methods are provided for effective management, prophylaxis, and/or treatment of neuroleptic malignant syndrome. This condition is a life-threatening, neurological disorder most often caused by an adverse reaction to neuroleptic or antipsychotic drugs, specifically those that affect the central dopaminergic system. Symptoms often include high fever, sweating, unstable blood pressure, stupor, muscular rigidity, and/or autonomic nervous system dysfunction. This condition can also be caused by drugs such as prochlorperazine, droperidol, or metoclopramide.

Within other exemplary embodiments of the invention, bicifadine formulations and methods are provided for effective management, prophylaxis, and/or treatment of malignant hyperthermia. Malignant hyperthermnia is often triggered in genetically predisposed patients by certain inhalation anesthetics, e.g. chloroform, ether, halothane, enflurane, isoflurane, sevoflurane, deflurane and depolarizing muscle relaxants (e.g. suxamethonium). Malignant hyperthermia often manifests as a hypermetabolic state that may involve tachycardia, hypercarbia, rigidity and fever.

By administering the antipyretic bicifadine formulations of the invention in a suitable prophylactic or therapeutic treatment protocol, subjects presenting with, or at elevated risk for, neuroleptic malignant syndrome or malignant hyperthermia can be effectively treated. Treatment of these conditions using the formulations and methods provided herein will reduce or prevent elevated temperatures in these subjects, and will often additionally substantially prevent or alleviate one or more of the above-identified symptoms associated with the subject condition as well.

Within yet additional exemplary embodiments of the invention, bicifadine formulations and methods are provided for effective management, prophylaxis, and/or treatment of various forms of “hot flashes” that occur in mammalian subjects. Hot flashes are most commonly associated with menopause, however, they may also be drug induced (for example by anti-estrogen compounds such as tamoxifen, toremifen and raloxifen), or triggered by removal of estrogen-producing tissues (e.g., after abdominal hysterectomy and bilateral salpingo-oopherectomy (Loprinzi et al., 2000). As used herein, the term “hot flash” refers to any sudden, typically brief, sensation of heat, which often appears to affect the entire body, and may further be accompanied by secondary symptoms, including sweating, palpitations, and/or red blotching of the skin.

In the exemplary case of menopausal hot flashes (i.e., menopausal, post-menopausal, and peri menopausal hot flashes) the antipyretic compositions and methods of the invention are effective to substantially prevent or alleviate one or more of the foregoing symptoms. Antipyretic effectiveness of the inventive compositions and methods in this context may be demonstrated, for example, by a reduction in the number of hot flashes experienced by test versus control subjects, wherein the number of hot flashes of treated menopausal subjects may be reduced, for example, to fewer than 5 per day, fewer than 3 per day, fewer than 2 per day, fewer than 1 per day, or eliminated altogether. Alternatively, effectiveness may be demonstrated by a number of other numerical evaluation and scale rating systems including, but not limited to, the Kupperman Menopausal Index, the Menopause Rating Scale, Montgomery-Asberg Depression Rating Scale, the Hamilton Anxiety Rating Scale and the Hamilton Depression Rating Scale. Using the Hamilton Depression Rating Scale, for example, a score of 10-13 indicates mild depression; 14-17 mild to moderate depression; >17 moderate to severe depression. In the Hamilton Anxiety Rating Scale, mild anxiety is 18-24, moderate anxiety is 25-29 and severe anxiety would be any number over 30. With the Kupperman Menopausal Index is an assessment system that involves grading major menopausal symptoms from 0 (not present) to 3 (severe) and using the total score to quantify severity symptoms. The symptoms include hot flashes, depression, headache, palpitations, joint pain, loss of concentration, sleep disturbance, profuse perspiration, nervousness and irritability.

Hyperthermia is also common in cancer patients, either through infection, tumor development (causing paraneoplastic fever), drugs (allergic or hypersensitivity reactions), blood product transfusion, and graft-versus-host disease (GVHD). Paraneoplastic fever, or fever caused by tumors, is particularly common in patients presenting with lymphoma and renal cell carcinoma. These and other subjects are effectively treated, prophylactically and/or therapeutically, by administering to the subject an antipyretic effective amount of bicifadine sufficient to prevent or reduce temperature elevation, as noted above, or to prevent or alleviate one or more related hyperthermic response(s) and/or one or more symptom(s) secondary or attendant to hyperthermia in the subject.

In addition to the foregoing exemplary conditions that are amenable to treatment using the methods and formulations of the invention, additional thermal homeostatic impairments and deficiencies which can be effectively prevented and/or alleviated using these methods and compositions include numerous other forms/conditions of fever and pyresis, including but not limited to, hyperthermia caused by or associated with bacterial, viral, fungal, protistan or other parasitic infection, injury, inflammation, disease, endocrine disorders, and stroke, among other conditions.

Within additional aspects of the invention, combinatorial antipyretic formulations and coordinate administration methods are provided which employ an effective amount of bicifadine and one or more additional active agent(s) that is/are combinatorially formulated or coordinately administered with bicifadine to yield an antipyretic composition or coordinate treatment method. Exemplary combinatorial formulations and coordinate treatment methods in this context employ bicifadine in combination with one or more additional antipyretic agents, and/or in combination with one or more additional analgesic agents. In additional combinatorial formulations and coordinate treatment methods, bicifadine is formulated or co-administered in combination with one or more secondary therapeutic agents used to treat symptoms which may accompany the hyperthermic conditions listed above. Thus, in all of the various embodiments described above there may be one or more secondary agent(s) included in an antipyretic bicifadine formulation or method. In the exemplary case of treating menopausal hot flashes, useful secondary agents in combinatorial formulations and coordinate treatment methods include, for example, secondary agents that are useful in the same treatment context, for example agents that alleviate hyperthermia, including but not limited to such agents as aspirin, ibuprofen, acetaminophen. In the case of treating menopausal hot flashes, and for other combinatorial treatment objectives, additional useful secondary agents in combinatorial formulations and coordinate treatment methods include agents that alleviate sleep disturbance, including but not limited to such agents as alprazolam, flurazepam, diazepam, zolpidem, zaleplon, and indiplon. In other methods and formulations for treating menopausal hot flashes, and for other combinatorial treatment objectives contemplated herein, useful secondary agents in combinatorial formulations and coordinate treatment methods include agents that alleviate cognitive dysfunction, including but not limited to such agents as tacrine, rivastigmine, galantamine, donepizil, and memantine In other methods and formulations for treating menopausal hot flashes, and for other combinatorial treatment objectives contemplated herein, useful secondary agents in combinatorial formulations and coordinate treatment methods include agents that alleviate mood disturbances and/or cognitive dysfunction, including but not limited to such agents as fluoxetine, paroxetine, venlafaxine, citalopram, desipramine, imipramine, deprenyl, tranylcypromine and phenelzine. In still additional methods and formulations for treating menopausal hot flashes, and for other combinatorial treatment objectives contemplated herein, useful secondary agents in combinatorial formulations and coordinate treatment methods include agents that alleviate night sweats and/or palpitations, including but not limited to such agents as propranolol and albuterol; and/or anxiety, such as diazepam, chlordiazepoxide, temazepam, alprazolam, triazolam; clonazepam; and buspirone.

Thus, in certain detailed embodiments the invention provides combinatorial antipyretic formulations comprising bicifadine and one or more additional agent(s) having antipyretic activity (wherein bicifadine and the one or more additional agent(s) having antipyretic activity are present in the combined formulation in antipyretically effective amounts, alone or in combination). In exemplary embodiments, combinatorial formulations of the invention comprise an antipyretic effective combination of bicifadine and a non-bicifadine antipyretic agent. The bicifadine and non-bicifadine antipyretic agents may each be present in an amount (i.e., singular dosage), that is antipyretically effective (i.e., which will alone elicit a detectable antipyretic response in the subject). Alternatively, the combinatorial formulation may comprise one or both of the bicifadine and non-bicifadine antipyretic agents in sub-therapeutic singular dosage amount(s), wherein the combinatorial formulation comprising both agents features a combined dosage of both agents that is collectively antipyretically effective. Thus, one or both of the bicifadine and non-bicifadine antipyretic agents may be present in the formulation, or administered in a coordinate administration protocol, at a sub-therapeutic dose, but collectively in the formulation or method they elicit a detectable antipyretic response in the subject.

Within these various combinatorial formulations, bicifadine may be combined in an antipyretic composition with one or more non-bicifadine antipyretic agents. Many such non-bicifadine antipyretic agents are known in the art and all such known agents are considered within the scope of these embodiments. Exemplary non-bicifadine antipyretic agents for use within these aspects of the invention include, but are not limited to, antipyretic NSAIDS; antipyretic narcotics; antipyretic antidepressants (such as monoamine oxidase inhibitors); antipyretic selective serotonin reuptake inhibitors; antipyretic selective noradrenaline reuptake inhibitors; and antipyretic serotonin and norepinephrine uptake inhibitors. Within more detailed embodiments, selected non-bicifadine antipyretic agents for use in combinatorial antipyretic formulations may include one or more of, or any combination of, the foregoing types of agent(s). Exemplary non-bicifadine antipyretics within these embodiments may include, but are not limited to, clonidine, venalfaxine, paroxetine, gabapentin, cycloheximide (Young et al., Cancer Res. 35:1218-1224, 1995), dexamethasone, melanocortins (Huang et al., J. Neurosci. 17:3343, 1997), nitric oxide and related nitrogen compounds, as well as many non-steroidal and steroidal anti-inflammatories (NSAIDs) that possess antipyretic activity (alone or in combination with bicifadine). Exemplary antipyretic NSAIDs in this context include, but are not limited to, ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate. Exemplary antipyretic steroidal anti-inflammatories in this context include, but are not limited to, cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, flucetonide, fludrocortisone acetate, betamethasone benzoate, chloroprednisone acetate, clocortolone acetate, descinolone acetonide, desoximetasone, dichlorisone acetate, difluprednate, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

Additional non-bicifadine antipyretic agents for use in this context may include various natural products and derivatives, including whole or processed materials, extracts, derivatives or synthetic analogs of natural products, including plant and animal products—for example including, but not limited to, vertebrate organ, tissue, cell, protein or peptide products, including vertebrate protein and peptide hormones, and plant products derived from such sources as soy, black cohosh, chaste tree berry, dong quai, ginseng, evening primrose oil, motherwort, red clover, licorice, and like sources.

Within other detailed embodiments the invention provides combinatorial antipyretic and analgesic dual activity formulations comprising bicifadine and one or more additional agent(s) having either or both antipyretic and/or analgesic activity. Within these combinatorial formulations and treatment methods, at least a portion of the antipyretic activity of the formulation or method is contributed by bicifadine. At the same time, all or a portion of the second, analgesic activity of the formulation may be contributed as well by the bicifadine. Accordingly, these dual efficacy, antipyretic and analgesic formulations and methods may include all of the combinatorial formulations and coordinate treatment methods identified above on the context of combinatorial antipyretic formulations (and some or all of the secondary, analgesic activity may come from the bicifadine and/or the non-bicifadine antipyretic which may also have dual function as an antipyretic and analgesic.

Nonetheless, in additional specific embodiments comprising a dual effective antipyretic and analgesic combinatorial formulation or coordinate treatment method, bicifadine is combined with one or more additional agent(s) having analgesic activity as a specific required secondary component of the formulation or method. The second, analgesically active agent will often be present or co-administered in the combined formulation or coordinate administration method in an analgesically effective amount. However, the additional, non-bicifadine analgesic and the bicifadine may each be present in sub-analgesic amounts (i.e., singular dosages), that are combinatorially or coordinately analgesically effective (i.e., which will only collectively elicit a detectable analgesic response in the subject). Alternatively, the combinatorial formulation may comprise one or both of the bicifadine and non-bicifadine analgesic agents in therapeutic singular dosage amount(s), wherein the combinatorial formulation (or coordinate administration method) elicits an enhanced analgesic response. In all cases, however, these combinatorial formulations and coordinate treatment methods are characterized in that the formulation and method elicits a significant, or detectable, antipyretic response in the subject, and at least a portion of the antipyretic activity of the formulation or method is attributable to the presence of the bicifadine.

Within these additional, dual activity antipyretic and analgesic combinatorial formulations, bicifadine may be combined in an antipyretic composition with one or more non-bicifadine analgesic agents. Many such non-bicifadine analgesic agents are known in the art and all such known analgesic agents are considered within the scope of these embodiments, regardless of whether they may also have individual or combinatorial (i.e., with bicifadine) antipyretic activity as discussed above. In addition, exemplary non-bicifadine analgesic agents for use within these combinatorial formulations and coordinate treatment methods include, but are not limited to, any one or combination of analgesic NSAIDs, acetominophen, all analgesic narcotics, ibuprofen, ketoprofen, flurbiprofen, fenoprofen, loxoprofen, suprofen, aluminoprofen, pranoprofen, piroxicam, pentazocine, aspirin, acetanilide, phenacetin, diclofenac, antipyrine, aminopyrine, phenyl salicylate, methyl salicylate, methenamine, carprofen, choline salicylate, salsalate, diflunisal, dihydroergotamine mesylate, ergotamine tartrate, indomethacin, meclofenamate, mefenamic acid, naproxen, oxyphenbutazone, phenylbutazone, sulindac, tolmetin, and/or cyclooxygenase 2 (COX 2) inhibitors.

To practice the coordinate administration methods of the invention, bicifadine is administered, simultaneously or sequentially, in a coordinate treatment protocol with one or more of the secondary therapeutic agents contemplated herein. Thus, in certain embodiments bicifadine is administered coordinately with a non-bicifadine antipyretic, a non-bicifadine analgesic, or any other secondary therapeutic agent contemplated herein, using separate formulations or a combinatorial formulation as described above (i.e., comprising both the bicifadine and non-bicifadine therapeutic agent). This coordinate administration may be done simultaneously, or sequentially in either order, and there may be a time period while only one or both (or all) active therapeutic agents individually and/or collectively exert their biological activities. A distinguishing aspect of all such coordinate treatment methods is that the bicifadine exerts at least some detectable antipyretic activity, which yields a favorable clinical response in conjunction with a secondary clinical response provided by the secondary therapeutic agent. Often, the coordinate administration of bicifadine with the secondary therapeutic agent will yield an enhanced antipyretic, analgesic or other therapeutic response beyond an antipyretic, analgesic or other therapeutic response elicited by the secondary therapeutic agent.

Within exemplary embodiments, bicifadine will be coordinately administered (simultaneously or sequentially, in combined or separate formulation(s)) with one or more secondary antipyretic, analgesic or other therapeutic agents, e.g., selected from non-bicifadine analgesics, non-bicifadine antipyretics, narcotics, anti-inflammatories, antidepressants, herbal derivatives, vitamins, NSAIDs, acetominophen, ibuprofen, ketoprofen, flurbiprofen, fenoprofen, loxoprofen, suprofen, aluminoprofen, pranoprofen, piroxicam, pentazocine, aspirin, acetanilide, phenacetin, diclofenac, antipyrine, aminopyrine, phenyl salicylate, methyl salicylate, methenamine, carprofen, choline salicylate, salsalate, diflunisal, dihydroergotamine mesylate, ergotamine tartrate, indomethacin, meclofenamate, mefenamic acid, naproxen, oxyphenbutazone, phenylbutazone, sulindac, tolmetin, and/or cox2 inhibitors. Antidepressants contemplated for use in the present invention include, but are not limited to monoamine oxidase inhibitors, selective serotonin reuptake inhibitors, tricyclic antidepressants, tetracyclic antidepressants, norepinephrine uptake inhibitors, selective noradrenaline reuptake inhibitors and serotonin and norepinephrine uptake inhibitors, including, for example, venlafaxine, paroxetine, and citalopram. Herbal derivatives contemplated for use in the present invention include, but are not limited to black cohosh, soy, chaste tree berry, dong quai, ginseng, evening primrose oil, motherwort, red clover and licorice. Additionally, vitamins such as vitamin E may be used in combinatorial compositions and coordinate administration methods of the present invention.

As noted above, in all of the various embodiments of the invention contemplated herein, the antipyretic methods and formulations may employ bicifadine in a variety of forms, including any one or combination of its pharmaceutically acceptable salts, polyrnorphs, solvates, hydrates, and/or prodrugs. In exemplary embodiments of the invention, bicifadine hydrochloride is employed within the therapeutic formulations and methods for illustrative purposes.

Bicifadine HCl, ((±)-1-(4-methylphenyl)-3-azabicyclo[3.1.0]hexane hydrochloride. DOV 220,075), also named racemic 1-(p-tolyl)-3-azabicyclo[3.1.0]hexane hydrochloride, is a non-narcotic analgesic is disclosed in U.S. Pat. No. 4,231,935 and U.S. Pat. No. 4,196,120. It is represented by the structural formula I, below:

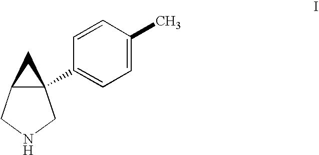

Bicifadine HCl also exists in at least two polymorphic crystalline forms, designated polymorph forms A and B (as described in U.S. patent application Ser. No. 10/702,397, herein incorporated by reference). Other polymorph forms of bicifadine hydrochloride may exist and are considered to be within this disclosure. Polymorphs are compounds with identical chemical structure but different internal structures. Additionally, many pharmacologically active organic compounds regularly crystallize incorporating second, foreign molecules, especially solvent molecules, into the crystal structure of the principal pharmacologically active compound forming pseudopolymorphs. When the second molecule is a solvent molecule, the pseudopolymorphs can also be referred to as solvates. All of these additional forms of bicifadine are likewise useful within the antipyretic methods and formulations of the invention.

Polymorph form A of bicifadine HCL can be formed by at least any of the methods disclosed in U.S. Pat. No. 4,231,935 and U.S. Pat. No. 4,196,120 (each incorporated herein by reference). Polymorph form B can be formed by any suitable method, including the methods disclosed in U.S. patent application Ser. No. 10/702,397, herein incorporated by reference. For example, polymorph B can be formed from polymorph form A through the application of kinetic energy, and through crystallization techniques. In one embodiment, kinetic energy in the form of agitating, stirring, grinding or milling can be applied to polymorph form A especially at low temperatures, generally from about −200° C. to about 50° C., in another embodiment from about −200° C. to about 35° C., in a further embodiment from about −200° C. to about 0° C. In another embodiment, polymorph B can be crystallized from a solution of polymorph A can be heated and allowed to cool for a sufficient amount of time to form polymorph B.

The polymorphs of bicifadine HCl may be characterized by their infrared spectra and/or their x-ray powder diffraction pattern. The X-ray powder diffraction (XRPD) analyses of polymorph forms A and B of racemic bicifadine hydrochloride were performed with a Shimadzu XRD-6000 X-ray powder diffractometer using Cu Ka radiation. The bicifadine was loaded onto the machine as a crystalline powder. The instrument was equipped with a fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A theta-two theta continuous scan at 3/min (0.4 sec/0.02° step) from 2.5 to 40°2θwas used. A silicon standard was analyzed to check the instrument alignment. Data were collected and analyzed using XRD-6000 v.4.1.

The X-ray powder diffraction pattern of polymorph form A of racemic bicifadine hydrochloride is given in terms of “d” spacing and relative intensities (I) is as follows (s=strong, m=medium, w=weak, v=very, d=diffuse) and these terms are set forth in Table 1 below, and the X-ray powder diffraction pattern of form B of bicifadine hydrochloride is set forth in Table 2 below:

TABLE 1
Peak Positions, d-Spacings, and Intensities for Polymorph
Form A Bicifadine Hydrochloride

| 2θ (deg) | d (Å) | I^{a} |
|---|---|---|
| 5.35 | 16.50 | Vs |
| 10.61 | 8.33 | Vs |
| 11.45 | 7.72 | W |
| 15.22 | 5.82 | W |
| 15.93 | 5.56 | W |
| 16.97 | 5.22 | W |
| 18.37 | 4.83 | W |
| 20.04 | 4.43 | Md |
| 20.26 | 4.38 | Md |
| 21.22 | 4.18 | M |
| 21.89 | 4.06 | S |
| 23.12 | 3.84 | Md |
| 23.54 | 3.78 | Wd |
| 26.63 | 3.34 | M |
| 27.83 | 3.20 | Wd |
| 28.32 | 3.15 | Wd |
| 30.67 | 2.91 | Wd |
| 32.03 | 2.79 | S |
| 37.57 | 2.39 | W |
| 38.20 | 2.35 | W |

^{a}S = strong, M = medium, W = weak, V = very, d = diffuse

TABLE 2
Peak Positions, d-Spacings, and Intensities for Polymorph
Form B Bicifadine Hydrochloride

| 2θ (deg) | d (Å) | I^{a} |
|---|---|---|
| 5.08 | 17.39 | Vs |
| 10.07 | 8.77 | S |
| 15.19 | 5.83 | S |
| 16.83 | 5.27 | S |
| 18.64 | 4.76 | Md |
| 18.76 | 4.73 | Md |
| 19.64 | 4.52 | W |
| 20.16 | 4.40 | M |
| 21.96 | 4.05 | M |
| 22.37 | 3.97 | S |
| 23.16 | 3.84 | W |
| 24.00 | 3.70 | W |
| 25.27 | 3.52 | D |
| 27.33 | 3.26 | Md |
| 27.74 | 3.21 | M |
| 29.00 | 3.08 | M |
| 30.43 | 2.93 | Md |
| 31.84 | 2.80 | Wd |
| 32.29 | 2.77 | W |
| 35.27 | 2.54 | Wd |
| 35.64 | 2.52 | W |

^{a}S = strong, M = medium, W = weak, V = very, D = diffuse

Table 1 and Table 2 represent the XRPD pattern of the peak positions of bicifadine hydrochloride form A and form B respectively having reduced particle size. The results in these tables demonstrate the difference between the XRPD patterns of form A and form B at a reduced particle size. However, there are key peaks at given angles in this pattern which identify polymorph form B of bicifadine hydrochloride and are typically present in XRPD pattern of polymorph form B irrespective of its particle size. These angles, expressed as 20 (deg), locating these major peaks which characterize the polymorph form B, using Cu Ka radiation, are: 5.08; 10.07; 20.16; 25.17; and 30.43

The infrared spectra were obtained for each of the samples using a Magna-IR 860® Fourier transform infrared (FT-IR) spectrophotometer (Thomas Nicolet) equipped with an Ever-Glo mid/far IR source, an extended range potassium bromide (KBr) beamsplitter, and a deuterated triglycine sulfate (DTGS) detector. The spectrophotometer measured the intensity of infrared light bands of each of the samples at given wavelengths. A diffuse reflectance accessory (the Collector ™, Thermo Spectra-Tech) was used for sampling. Each spectrum represents 256 co-added scans collected from 400-4000 cm⁻¹ at a spectral resolution of 4 cm⁻¹. Sample preparation consisted of placing the sample of powder containing crystals in either polymorph form A or form B into a 13-mm diameter cup and leveling the material with a frosted glass slide. A background data set was acquired with an alignment mirror in place. The reflectance R is the ratio, at a given wavenumber, of the light intensity of the sample/light intensity of the background set. A Log 1/R(R=reflectance) spectrum acquired by taking a ratio of these two data sets (the sample and the background light intensities) against each other. The infrared spectrum of polymorph A or racemic bicifadine hydrochloride as a dry crystalline powder, as provided in Table 3, showed the indicated main peaks which characterized this polymorph. The infrared spectrum of polymorph B of racemic bicifadine hydrochloride in dry crystalline powder, as provided in Table 4, showed the indicated main peaks which characterize this polymorph.

TABLE 3
Infrared Peak Positions For Polymorph Form A Bicifadine Hydrochloride.
All values in wavenumbers (cm⁻¹)

| | |
|---|---|
| 3949 | 1088 |
| 2923 | 1068 |
| 2431 | 1050 |
| 2280 | 900 |
| 2091 | 825 |
| 1895 | 781 |
| 1790 | 714 |
| 1595 | 689 |
| 1522 | 652 |
| 1430 | 574 |
| 1376 | 533 |
| 1233 | 437 |
| 1130 | |

TABLE 4
Infrared Peak Positions for Polymorph Form B Bicifadine Hydrochloride.
All values in wavenumbers (cm⁻¹)

| | |
|---|---|
| 3185 | 1111 |
| 2769 | 1022 |
| 2437 | 963 |
| 2276 | 904 |
| 2108 | 891 |
| 1908 | 856 |
| 1804 | 818 |
| 1658 | 783 |
| 1596 | 719 |
| 1518 | 684 |
| 1453 | 660 |
| 1403 | 637 |
| 1343 | 580 |
| 1305 | 532 |
| 1274 | 475 |
| 1209 | 422 |
| 1131 | |

Table 3 and Table 4 provide the complete patterns of the infrared peak positions with respect to polymorph form A and polymorph form B of bicifadine hydrochloride respectively. However, there are certain key peaks, within this pattern, which are associated with polymorph form B of bicifadine hydrochloride and are sufficient to characterize this polymorph. These peaks, expressed in wavenumbers (cm⁻1), are: 2108; 891; 856; 719; and 660.

Effective dosages of bicifadine may comprise any crystalline polymorphic or amorphous form of the compound, or mixture(s) thereof. For example, the effective dosage of bicifadine in a therapeutic formulation as provided herein may comprise substantially pure bicifadine HCl polymorph “form A“, essentially pure polymorph “form B”, or any mixture of polymorph form A and B. In certain embodiments, the composition may contain from about 10% to 98% polymorph form B. In other embodiments there may be present in the formulation greater than about 50% polymorph form B, greater than about 75% polymorph B, or greater than about 90% polymorph B.

Suitable routes of administration for antipyretic and related, combinatorial compositions of the invention comprising bicifadine include, but are not limited to, oral, buccal, nasal, aerosol, topical, transdermal, mucosal, injectable, slow release, controlled release, iontophoresis, sonophoresis, and including all other conventional delivery routes, devices and methods. Injectable methods include, but are not limited to, intravenous, intramuscular, intraperitoneal, intraspinal, intrathecal, intracerebroventricular, intraarterial, subcutaneous and intranasal routes.

The compositions of the invention for treating hyperthermia can further include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives/agents will often be biologically inactive and can be administered to patients without causing deleterious interactions with the active agent.

If desired, the bicifadine can be administered in a controlled release form by use of the hydrophilic slow release polymer, hydroxypropyl methyl cellulose, in an oral unit dosage or other suitable form. Any hydrophilic slow release polymer can be utilized, such as hydroxypropyl methyl cellulose polymer having a viscosity in the range of about 100 cps to about 100,000 cps.

Commonly the antipyretic bicifadine compositions and combinatorial formulations of the invention will be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets, which may be prepared by any conventional method of preparing pharmaceutical oral unit dosage forms can be utilized in preparing oral unit dosage forms. Oral unit dosage forms, such as tablets, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants.

Additional antipyretic bicifadine compositions and combinatorial formulations of the invention may be prepared and administered in any of a variety of inhalation or nasal delivery forms known in the art. Devices capable of depositing aerosolized bicifadine formulations in the sinus cavity or pulmonary alveoli of a patient include metered dose inhalers, nebulizers, dry powder generators, sprayers, and the like. Suitable formulations, wherein the carrier is a liquid, for administration, as for example, a nasal spray or as nasal drops, may include aqueous or oily solutions of bicifadine and any additional active or inactive ingredient(s).Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the compositions in a suitable liquid carrier.

Yet additional compositions and methods are provided for topical administration of bicifadine and/or secondary therapeutic agents for antipyretic or coordinate therapeutic use. Topical compositions may comprise bicifadine and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise bicifadine dissolved or dispersed in a portion of a water or other solvent or liquid to be incorporated in the topical composition or delivery device.

Yet additional bicifadine compositions and combinatorial formulations are provided for parenteral administration, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Bicifadine antipyretic formulations may also include polymers for extended release following parenteral administration. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, antipyretic formulations may comprise bicifadine and one or more optional secondary therapeutic agent(s) encapsulated in microcapsules, microparticles, or microspheres, prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions.

The above disclosure generally describes the present invention. A more complete understanding can be obtained by reference to the following examples. These examples are described solely for purposes of illustration and are not intended to limit the scope of the invention. Although specific terms have been employed herein, such terms are intended in a descriptive sense and not for purposes of limitations.

### EXAMPLES

Utilizing in vitro and in vivo analytical methods, it is demonstrated herein that bicifadine HCl ((±)-1-(4-methylphenyl)-3-azabicyclo[3.1.0]hexane hydrochloride, DOV 220,075) possesses significant antipyretic activity. This novel activity and use may be related to the ability of bicifadine to modulate noradrenergic and serotonergic neurotransmission by a combination of interactions with α₁ and α₂ adrenergic, and 5-HT_{2A} receptors, as well as inhibition of norepinephrine re-uptake.

Insights into the potential mechanism by which bicifadine HCl expresses its antipyretic action were provided by biochemical assays (Table 5). While bicifadine HCl did not inhibit prostoglandin synthesis, it was capable of occupying binding sites on both the α₁ and α₂ adrenergic receptors. In addition, bicifadine HCl significantly inhibited radioligand binding to the 5-HT_{2A} serotonin receptor. All three of these receptor subtypes are involved in thermoregulatory processes in the central and peripheral nervous system. The interaction of bicifadine HCl with this combination of receptors may contribute to the antipyretic profile of bicifadine HCl. Moreover, the side-effect profile may be significantly reduced compared to other antipyretics. Because bicifadine HCl does not inhibit prostaglandin synthesis, it should not produce gastrointestinal lesions, as do NSAIDS. In addition, the ability to inhibit norepinephrine uptake suggests that bicifadine is less likely to induce vasodilatation and hypotension, unlike selective al antagonists. This may be supported by the observation that bicifadine HCl did not cause significant alterations in blood pressure in human subjects.

### Example I

### Preparation of 1-(p-tolyl)-3-azabicyclo[3.1/0]hexane hydrochloride

230 ml of thionyl chloride was added to 120 g of p-tolylacetic acid and the solution was allowed to stand at room temperature for 2 hours, after which it was warmed to 60° C. for 1 hour. To this solution 285 g of N-bromosuccinimide and 10 drops of 48% hydrobromic acid were added and the mixture was refluxed on a 90° C. oil bath for 1 hour. An additional 90 ml of thionyl chloride was then added and refluxing continued for an additional 45 minutes. The resulting mixture was distilled under reduced pressure to remove 250 ml of thionyl chloride, and the residual liquid was poured into 500 ml of cold methanol with stirring and ice cooling over 15 minutes. This solution was evaporated under reduced pressure to give a dark oil which was then dissolved in 100 ml of chloroform. The solution was washed with 500 ml of water, dried over magnesium sulfate and filtered. The filtrate was evaporated under reduced pressure to give a dark oil which was distilled to give 94 g of bromoester as a pale yellow liquid, bp. 115°-120° C. (0.05 mm). The pale yellow liquid was then reacted with methyl acrylate-sodium hydride in ether to give dimethyl cis-1-(p-tolyl)-1,2-cyclopropanedicarboxylate, mp 58°-59° C. Hydrolysis with 1 N potassium hydroxide, followed by acidification with 1N hydrochloric acid, yielded cis-1-(p-tolyl)-1,2-cyclopropanedicarboxylic acid as colorless crystals, mp 188°-190° C. A 5.7 g portion of this diacid and 2.02 g of urea in 200 ml of xylene was refluxed for 22 hours, cooled, diluted with benzene and washed with water. The organic layer was diluted with chloroform, dried, concentrated under reduced pressure, and recrystallized from ethyl acetate and petroleum ether to give 1-(p-tolyl)-1,2-cyclopropanedicarboximide as pale yellow crystals, mp 82°-85° C.

To a mixture of 20.1 g of this imide in 600 ml of benzene was added 160 ml of sodium bis(2-methoxyethoxy)aluminum hydride and the reaction was run, after which excess reagent was decomposed with 160 ml of 10 N sodium hydroxide. The benzene layer was washed with water, dried over magnesium sulfate and filtered. The filtrate was evaporated under reduced pressure to give a dark oil which was dissolved in ether, and then dry hydrogen chloride was bubbled into the solution. The resultant precipitate was collected by filtration and recrystallized from acetonitrile-methanol to give 12.1 g of 1-(p-tolyl)-3-azabicyclo[3.1.0]hexane hydrochloride as pale tan plates, mp 207°-208° C.

### Example II

### Preparation of (+)-1-(p-Tolyl)-3-azabicyclo[3.1.0]hexane hydrochloride

A solution of 94.8 g of racemic-1-(p-tolyl)-1,2-cyclopropanedicarboxylic acid and 73.8 g of (−)-α-(1-naphthyl)ethylamine in 300 ml of tetrahydrofuran was diluted with 300 ml of ethyl ether and was allowed to stand at room temperature until crystallization is complete. The mixture is filtered and the crystals which are collected were washed with cold tetrahydrofuran to give 4.95 g of a salt comprised of one molar equivalent of (+)-1-(p-tolyl)-1,2-cyclopropanedicarboxylic acid and one molar equivalent of (−)-α-(1-naphthyl)ethylamine. The salt was shaken with sodium hydroxide solution and ether. The aqueous phase was acidified with 12 N hydrochloric acid and the product was collected by filtration to give 26.0 g of (+)-1-(p-tolyl)-1,2-cyclopropanedicarboxylic acid as colorless crystals, [α]_{D}CH₃OH=+192°.

15.0 g portion of (+)-1-(p-tolyl)-1,2-cyclopropanedicarboxylic acid, 6.6 g of urea and 500 ml of xylene is refluxed and stirred for 5 hours. The reaction mixture was then filtered hot and the filtrate was evaporated under reduced pressure to give (+)-1-(p-tolyl)-1,2-cyclopropanedicarboximide as colorless crystals, m.p. 148°-155° C.

A 14 g portion of the above product was mixed with 420 ml of benzene and 112 ml of sodium bis(2-methoxyethoxy)aluminum hydride (70% benzene solution) was added over a 15 minute period with stirring. After refluxing for 1½ hours the mixture was cooled and 160 ml of 10 N sodium hydroxide was added. The organic layer was dried over sodium sulfate, filtered and evaporated to an oil. The oil was dissolved in ether and hydrogen chloride gas was bubbled in. The solid which forms was recrystallized from acetonitrile giving (+)-1-(p-tolyl)-3-azabicyclo[3.1.0]hexane hydrochloride as colorless crystals, m.p. 208°-210.5° C., [α]_{D}CH₃OH=+64.5°.

### Example III

### Conversion of Racemic Bicifadine Hydrochloride to Polyform B

Racemic bicifadine hydrochloride as a mixture of polymorphic forms A and B, was added to isopropyl alcohol in a sufficient quantity to form a slurry. The slurry was subjected to agitation, such as mixing, at a temperature less than 30° C. The product was isolated by filtration and dried at 50° C. in vacuo until loss on drying of <1% was achieved. The material produced was bicifadine hydrochloride polymorphic form B.

### Example IV

### Conversion of Racemic Bicifadine Hydrochloride to Polyform B

Twenty grams of racemic bicifadine hydrochloride as a mixture of polymorphic forms A and B were added to 50 ml of isopropyl alcohol to form a slurry. The slurry was stirred for 24 hours at a temperature of about 30° C. The product was isolated by filtration and dried in vacuo. The material produced was purified bicifadine hydrochloride polymorphic form B.

### Example V

### Demonstration of Antipyretic Actions of Bicifadine HCL in Rats

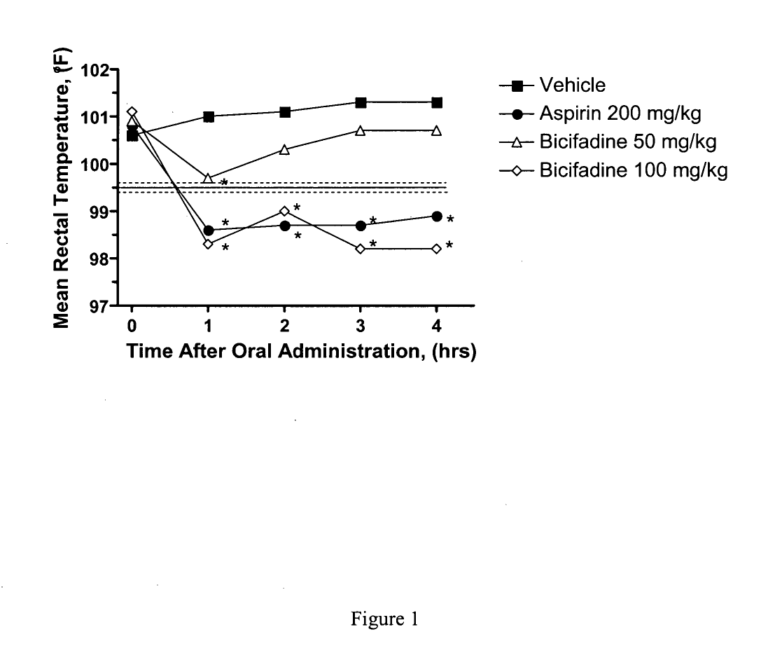

The antipyretic actions of bicifadine HCl were demonstrated in the yeast-induced pyresis model (Sofia et al., 1977) using rat subjects accepted in the art as a predictive model for antipyretic activity in humans, and the results are depicted in FIG. 1. Groups of 6 rats were injected subcutaneously with saline solution or 40% brewers' yeast 18 hours prior to oral administration of bicifadine HCl or aspirin. The yeast was suspended in 0.85% saline and doses in a volume of 10 ml/kg. Rectal temperatures were recorded with a YSI 43TA telethermometer just prior to oral drug administration, and hourly for 3-4 hours. The post-treatment temperatures were compared to the initial temperatures of each group. Control animals were treated with vehicle and tested concurrently. In FIG. 1, the solid line represents the mean rectal temperature of normal rats (99.5° F.) and the standard error of that measure (0.1° F.) is indicated by the dashed lines. The asterix (*) indicates temperatures significantly different from pre-treatment rectal temperature at t=0, P<0.05, Student's t-test.

The results shown in FIG. 1 indicate that rats treated with brewers' yeast 18 hours prior to drug administration develop a febrile response, manifested as a mean rectal temperature of 101.1° F. In contrast, normal rats treated only with vehicle have a rectal temperature of 99.5° F., 1.6° F. lower than the yeast-treated rats. Administration of 50 mg/kg bicifadine HCl (PO, σ) significantly reduced rectal temperature by the first hour after administration. Not only did 100 mg/kg bicifadine HCl (PO, υ) decrease rectal temperature to levels at or below normal, but it maintained this antipyretic effect for a full 4 hours. Moreover, bicifadine HCl was a more potent antipyretic than aspirin (200 mg/kg, PO, λ) in this assay. Despite its efficacy as an antipyretic, bicifadine HCl did not consistently lower the rectal temperature of normal rats (Table 5), thus suggesting that the bicifadine HCl does not induce hypothermia.

TABLE 5
Effect of bicifadine on average body temperature of normal rats.

| | Mean Rectal Temperature (° F.) | | | | |
|---|---|---|---|---|---|
| | at Indicated Hours Post-Treatment | | | | |
| Treatment | 0 | 1 | 2 | 3 | 4 |
| Vehicle | 99.3 | 99.6 | 99.3 | 99.5 | 99.6 |
| Bicifadine HCl, | 98.9 | 97.9* | 98.3 | 98.2 | 97.5 |
| 100 mg/kg PO | | | | | |

*Significantly different from temperature at t = 0 hrs, P < 0.05, Student's t-test. N = 6 for each group.

### Example VI

### Effect of Bicifadine HCl on Normal Human Temperature

Normal humans received either a placebo or 600 mg of bicifadine HCl (PO). Oral temperature readings were taken as part of an assessment of vital signs before and after administration of a single dose. Despite its efficacy as an antipyretic, bicifadine HCl did not consistently lower normal human oral temperature (Table 6), obviating the concern that it could induce hypothermia.

TABLE 6
Effect of bicifadine on mean body temperature of normal human subjects.

| | Oral Temperature (° F.) at | | | | | | | | |
|---|---|---|---|---|---|---|---|---|---|
| | Indicated Times of Treatment | | | | | | | | |
| Treatment | Pre-Dose | 0.5 hr | 1 | 2 | 3 | 4 | 5 | 6 | Follow-up |
| Placebo | 98.0 | 98.0 | 98.1 | 98.2 | 98.3 | 98.4 | 98.3 | 98.4 | 97.8 |
| Bicifadine | 98.1 | 98.1 | 98.1 | 98.1 | 98.0 | 98.1 | 98.2 | 98.2 | 97.8 |
| HCl, 600 mg | | | | | | | | | |
| PO | | | | | | | | | |

Oral temperature readings were taken as part of an assessment of vital signs before and after administration of a single dose of bicifadine to normal humans. The placebo group consisted of 150 subjects, while the bicifadine HCl group consisted of 151 individuals.

Further insight into the mechanism by which bicifadine HCl exerts its novel antipyretic activity was obtained by biochemical assays. Bicifadine HCl does not inhibit prostaglandin synthesis, as do NSAIDS. This was determined in assays using the rate limiting enzyme for prostaglandin synthesis, recombinant human cyclooxygenasel (COX₁) and cyclooxygenase2 (COX₂) expressed in Sf9 cells using a modified technique of Glaser et al (1995). These cells were incubated with arachidonic acid as the substrate (4, and 2 μM, respectively) and 0.1, 1, or 10 μM concentrations of bicifadine HCl in a suitable assay buffer. The assay mixture was incubated (10 min at 25° C. for COX₁, 5 min-at 22° C. for COX₂), then stopped and the amount of reaction product (PGE₂) formed was measured by an enzyme-linked immunosorbent assay. Assays were also performed using the COX₁ inhibitor diclofenac and the COX₂ inhibitor NS398 as reference agents. While the reference agents inhibited COX₁ and COX₂ activity with IC₅₀ values of 6.6 nM and 68 nM, respectively, bicifadine HCl had no significant inhibitory effect on PGE₂ synthesis by either enzyme.

For the α₁ adrenergic receptor, the ability of bicifadine to inhibit the binding of [³H]prazosin to receptors in a rat cerebral cortex preparation was investigated using a modification of the technique of Greengrass and Bremner (1979). The rat cortex preparation was incubated with a 0.25 nM concentration of [³H]prazosin for 60 min at 22° C. with either 0.1, 0.3, 1, 3, or 10 μM concentrations bicifadine HCl. Nonspecific binding was determined using 0.5 μM unlabelled prazosin. At the end of 60 min, the assay was terminated by vacuum filtration and the amount of radioactivity deposited on the filter measured by scintillation counting. Bicifadine HCl bound to the cc adrenergic receptor with low affinity (Kᵢ=1 μM), while the reference agent, prazosin bound with an affinity (Kᵢ) of 0.15 nM.

The above observations indicate that the side-effect profile attending the use of bicifadine as an antipyretic is significantly narrowed and reduced compared to other antipyretics. Because bicifadine HCl does not inhibit prostaglandin synthesis, its administration within the methods and compositions of the invention will not produce substantial or unacceptable occurrence of gastrointestinal discomfort, inflammation and/or lesions in mammalian subjects, which will be prevented or will occur at highest levels that are below levels observed for other antipyretics. For example, the rate of occurrence and/or severity of these side effects following administration of an antipyretic effective dose of bicifadine will be below, often 95% or less, 75% or less, 50% or less, 25-30% or less, and as low as 5-10% or less, compared to the rate of occurrence and/or severity of these side effects following administration of an antipyretic effective dose of an antipyretic NSAID or other conventional antipyretic as described above. In addition, the low affinity of bicifadine HCl for the α₁ receptor renders the bicifadine formulations and methods of the invention safer in terms of a comparably reduced or eliminated occurrence of vasodilation, hypotension and other related adverse symptoms elicited by selective α₁ antagonists. This improved characteristic of the inventive compositions and methods herein is evinced by the finding that bicifadine HCl did not cause significant alterations in blood pressure in human subjects. In selected embodiments of the invention, the rate of occurrence and/or severity of vasodilation and/or hypotension following administration of an antipyretic effective dose of bicifadine will be below, often 95% or less, 75% or less, 50% or less, 25-30% or less, and as low as 5-10% or less, compared to the rate of occurrence and/or severity of these side effects following administration of an antipyretic effective dose of a selective al antagonist.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

Aronoff, D. M., Neilson, E. G. Antipyretics: Mechanisms of action and clinical use in fever suppression. Am. J. Med. 111: 304-315, 2001.
Bhanushali, M. J., Tuite ,P. J. The evaluation and management of patients with neuroleptic malignant syndrome. Neurol. Clin. 22:389-411, 2004.
Blessing, W. W., and Seaman, B. 5-Hydroxytrptamine_{2A} receptors regulate sympathetic nerves constricting the cutaneous vascular bed in rabbits and rats. Neuroscience 117:939-948, 2003.
Birmes P, Coppin D, Schmitt L, Lauque D. Serotonin syndrome: a brief review. CMAJ 168:1439-42, 2003.
Cannon B., Nedergaard J. Brown adipose tissue: function and physiological significance. Physiol. Rev. 84: 277-359, 2004.
Frank S M, Raja S N, Wu P K, el-Gamal N. Alpha-adrenergic mechanisms of thermoregulation in humans. Ann. NY Acad. Sci. 813:101-110, 1997.
Fricchione, G., Mann, S., Caroff, S. Catatonia, lethal catatonia and neuroleptic malignant syndrome. Psychiatr. Ann. 30: 347-355, 2000.
Glaser, K., Sung, M. L., O'Neill, K., Belfast, M., Hartman, D., Carlson, R., Kreft, A., Kubrak, D., Hsiao, C. L., Weichman, B. Etodolac selectively inhibits human prostaglandin G/H synthase 2 (PGHS-2) versus human PGHS-1. Eur J Pharmacol. 281:107-11, 1995.
Granneman, J. G., Burnazi, M., Zhu, Z., Schwamb, L. A., White adipose tissue contributes to UCP1-independent thermogenesis. Am. J. Physiol. Endocrinol. Metab. 285:E1230-6, 2003.
Greengrass, P., Bremner, R. Binding characteristics of ³H-prazosin to rat brain alpha-adrenergic receptors. Eur J Pharmacol. 55:323-6, 1979.
Greisman, L. A., Mackowiak, P. A. Fever: beneficial and detrimental effects of antipyretics. Curr. Opin. Infect. Dis. 15: 241-245, 2002.
Halloran, L. L., Bernard, D. W. Management of drug-induced hyperthermia. Curr. Opin. Pediatr. 16:211-215, 2004.
Huang Q. H., Entwistle, M. L., Alvaro, J. D., Duman, R. S., Hruby, V. J., Tatro, J. B. Antipyretic role of endogenous melanocortins mediated by central melanocortin receptors during endotoxin-induced fever. J Neurosci. 17:3343-51, 1997.
Lazarus, A., Mann, S. C., Caroff, S. N. The neuroleptic malignant syndrome and related conditions. American Psychiatric Press, Inc., Washington, D.C., 1989.
Loprinzi C L, Zahasky K M, Sloan J A, Novotny P J, Quella S K, Tamoxifen-induced hot flashes. Clin. Breast Cancer 2000 April;1(1):52-6
Mackowiak, P. A., Normal “body” temperature. In: Mackowiak P. A., ed. Fever: Basic Mechanisms and Management. 2^{nd} ed. Philadelphia: Lippincott-Raven; 1997, pp 207-213.
Marshall, H. W., Stoner, H. B. Catecholaminergic alpha-receptors and shivering in the rat. J. Physiol. 292: 27-34, 1979.
Nisijima, K., Yoshino, T., Yui, K., Katoh, S. Potent serotonin (5-HT)_{2A} receptor antagonists completely prevent the development of hyperthermia in an animal model of the 5-HT syndrome. Brain Res. 890: 23-31, 2001.
Sofia, R. D., Diamantis, W., Ludwig, B. J. Pharmacological properties of 2,3-dihydro-9H-isoxozolo[3.2-b]quinozolin-9-one (W-2429), Part I: Analgesic, antipyretic and antiedema activity, Arzneim.-Forsch./Drug Res. 27:770-782, 1977.
Sprague, J. E, Banks, M. L., Cook, V. J., Mills, E. M. Hypothalamic-pituitary-thyroid axis and sympathetic nervous system involvement in the hyperthermia induced by 3,4-methylenedioxymethamphetamine (Ecstasy). J. Pharmacol. Exp. Ther. 305: 159-166, 2003.
Young C. W., Dowling M. D. Jr.Antipyretic effect of cycloheximide, and inhibitor of protein synthesis, in patients with Hodgkin's disease or other malignant neoplasms. Cancer Res. 35:1218-24, 1975.

We claim:

1. A method for preventing or treating hyperthermia in a mammalian subject comprising administering an antipyretic effective amount of bicifadine to said subject.

2. The method of claim 1, wherein the bicifadine comprises bicifadine hydrochloride.

3. The method of claim 2, wherein the bicifadine comprises a polymorph A, polymorph B, or a mixture of A and B polymorphs of bicifadine hydrochloride.

4. The method of claim 1, further comprising administering a second antipyretic agent to said subject.

5. The method of claim 4, wherein the second antipyretic agent is administered to said subject in a combined formulation with said bicifadine.

6. The method of claim 4, wherein the second antipyretic agent is administered to said subject in a coordinate administration protocol, simultaneously with, prior to, or after, administration of said bicifadine to the subject.

7. The method of claim 4, wherein the second antipyretic agent is selected from antipyretic NSAIDS; antipyretic narcotics; antipyretic antidepressants, antipyretic selective serotonin reuptake inhibitors, antipyretic selective noradrenaline reuptake inhibitors, and antipyretic serotonin and norepinephrine uptake inhibitors.

8. The method of claim 4, wherein the second antipyretic agent is selected from clonidine, venlafaxine, paroxetine, gabapentin, cycloheximide, dexamethasone, melanocortins, and agents that increase nitric oxide levels, such as phosphodiesterase inhibitors (including but not limited to sildenafil, vardenafil, and theophylline) and nitric oxide donors (including but not limited to nitroglycerine, arginine).

9. The method of claim 4, wherein the second antipyretic agent is an antipyretic non-steroidal anti-inflammatory agent (NSAID).

10. The method of claim 9, wherein the antipyretic NSAID is selected from ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

11. The method of claim 4, wherein the second antipyretic agent is an antipyretic steroidal anti-inflammatory agent.

12. The method of claim 4, wherein the antipyretic steroidal anti-inflammatory agent is selected from cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, flucetonide, fludrocortisone acetate, betamethasone benzoate, chloroprednisone acetate, clocortolone acetate, descinolone acetonide, desoximetasone, dichlorisone acetate, difluprednate, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

13. The method of claim 4, wherein the second antipyretic agent is a natural product.

14. The method of claim 4, wherein the natural product is selected from vertebrate organ, tissue, cell, protein and peptide products, vitamins, and plant products.

15. The method of claim 14, wherein the plant product is selected from soy, black cohosh, chaste tree berry, dong quai, ginseng, evening primrose oil, motherwort, red clover, and licorice.

16. The method of claim 1, further comprising administering a non-bicifadine analgesic agent in an amount effective in combination with said bicifadine to elicit an analgesic response in said subject.

17. The method of claim 16, wherein the non-bicifadine analgesic agent is administered to said subject in a combined formulation with said bicifadine.

18. The method of claim 16, wherein the non-bicifadine analgesic agent is administered to said subject in a coordinate administration protocol, simultaneously with, prior to, or after, administration of said bicifadine to the subject.

19. The method of claim 18, wherein the non-bicifadine analgesic agent is selected from analgesic NSAIDs, analgesic narcotics, acetominophen, ibuprofen, ketoprofen, flurbiprofen, fenoprofen, loxoprofen, suprofen, aluminoprofen, pranoprofen, piroxicam, pentazocine, aspirin, acetanilide, phenacetin, diclofenac, antipyrine, aminopyrine, phenyl salicylate, methyl salicylate, methenamine, carprofen, choline salicylate, salsalate, diflunisal, dihydroergotamine mesylate, ergotamine tartrate, indomethacin, meclofenamate, mefenamic acid, naproxen, oxyphenbutazone, phenylbutazone, sulindac, tolmetin, and/or cyclooxygenase 2 (COX2) inhibitors.

20. The method of claim 1, wherein said subject is a menopausal female and wherein said method effectively prevents or alleviates peri menopausal, menopausal, or post menopausal hot flashes.

21. The method of claim 20, further comprising administering a second therapeutic agent effective for treating one or more menopause-related symptom(s) or condition(s) in said subject selected from hot flashes, depression, headache, palpitations, joint pain, loss of concentration, cognitive dysfunction, mood disturbance, sleep disturbance, profuse perspiration, night sweats, palpitations, nervousness and irritability.

22. The method of claim 21, wherein the second therapeutic agent is an antidepressant.

23. The method of claim 21, wherein the second therapeutic agent is an anxiolytic agent.

24. The method of claim 1, further comprising administering an antidepressant to said subject.

25. The method of claim 24, wherein the antidepressant is selected from monoamine oxidase inhibitors, selective serotonin reuptake inhibitors, tricyclic antidepressants, tetracyclic antidepressants, norepinephrine uptake inhibitors, selective noradrenaline reuptake inhibitors, and serotonin uptake inhibitors, and norepinephrine uptake inhibitors.

26. The method of claim 25, wherein the antidepressant is selected from venlafaxine, paroxetine, and citalopram.

27. The method of claim 1, wherein said antipyretic effective amount comprises between about 20 mg to about 1,200 mg of bicifadine.

28. The method of claim 27, wherein said antipyretic effective amount comprises between about 25 mg to about 500 mg of bicifadine.

29. The method of claim 28, wherein said antipyretic effective amount comprises between about 50 mg to about 250 mg of bicifadine.

30. The method of claim 1, wherein the administration of bicifadine is antipyretically effective to lower an elevated temperature in said subject by about 0.5 5° F. to about 5° F.

31. The method of claim 1, wherein the administration of bicifadine is antipyretically effective to lower an elevated temperature in said subject by about at least 1 to 2° F.

32. A method of controlling body temperature in a mammalian subject to reduce or prevent elevation of body temperature comprising administering to the subject an antipyretic effective amount of bicifadine to said subject.

33. The method of claim 32, wherein the method is effective to prevent or alleviate a condition of hyperthermia, fever, or pyresis in said subject.

34. The method of claim 33, wherein the hyperthermia, fever, or pyresis is associated with inflammation, infection, post surgical reaction, cancer or stroke in said subject.

35. The method of claim 32, wherein the hyperthermia, fever, or pyresis is associated malignant hyperthermia in said subject.

36. The method of claim 32, wherein the hyperthermia, fever, or pyresis is drug induced.

37. The method of claim 36, wherein the drug inducing the hyperthermia is chloroform, ether, halothane, enflurane, isoflurane, sevoflurane, deflurane, depolarizing muscle relaxants, suxamethonium, prochlorperazine, droperidol, or metoclopramide.

38. The method of claim 32, wherein the hyperthermia, fever, or pyresis is associated with a hot flash condition in said subject.

39. The method of claim 38, wherein the hot flash condition is associated with menopause, peri menopause, post menopause, anti-estrogen therapy, surgical removal of estrogen-producing tissue, or radiation therapy in said subject.

40. The method of claim 38, wherein the hot flash condition is drug induced.

41. The method of claim 32, wherein the hyperthermia, fever, or pyresis is associated with drug exposure or overdose in said subject.

42. The method of claim 41, wherein the drug is MDMA, clomipramine or trazadone.

43. The method of claim 42, wherein the drug is MDMA.

44. The method of claim 32, wherein the subject exhibits serotonin syndrome.

45. The method of claim 32, wherein the effective amount is between about 70 to 1800 mg per day.

46. The method of claim 32, wherein said antipyretic effective amount is between about 20 mg to about 1,200 mg of bicifadine.

47. The method of claim 46, wherein said antipyretic effective amount is between about 25 mg to about 500 mg of bicifadine.

48. The method of claim 47, wherein said antipyretic effective amount is between about 50 mg to about 250 mg of bicifadine.

49. The method of claim 32, wherein the administration of bicifadine is antipyretically effective to lower an elevated temperature in said subject by about 0.5 5° F. to about 5° F.

50. The method of claim 32, wherein the administration of bicifadine is antipyretically effective to lower an elevated temperature in said subject by about at least 1 to 2 ° F.

51. A method of treating one or more symptoms of peri menopause, menopause or post menopause comprising administering to a mammalian female subject an effective amount of bicifadine.

52. The method of claim 51, wherein said one or more symptoms of peri menopause, menopause or post menopause is/are selected from hot flashes, depression, headache, palpitations, joint pain, loss of concentration, cognitive dysfunction, mood disturbance, sleep disturbance, profuse perspiration, night sweats, palpitations, nervousness and irritability.

53. The method of claim 51, which is effective to prevent or reduce menopausal, post-monopausal, or peri menopausal hot flashes in said subject.

54. The method of claim 51, which is effective to reduce a Hamilton Depression Rating Scale of said subject to below a value of less than about 15, and/or to reduce a Kupperman Menopausal Index of said subject to a value of less about 15.

55. The method of claim 51, further comprising administering an antidepressant to said subject.

56. The method of claim 51, further comprising administering a secondary therapeutic agent effective for treating one or more menopause-related symptom(s) or condition(s) in said subject selected from hot flashes, depression, headache, palpitations, joint pain, loss of concentration, cognitive dysfunction, mood disturbance, sleep disturbance, profuse perspiration, night sweats, palpitations, nervousness, and irritability.

57. A composition for preventing or alleviating hyperthermia in a mammalian subject comprising an antipyretic effective amount of bicifadine or a pharmaceutically-acceptable salt, solvate, polymorph or prodrug thereof.

58. A composition for treating hyperthermia in a mammalian subject comprising bicifadine and a second antipyretic agent.

59. The composition of claim 58, wherein the second antipyretic agent is selected from antipyretic NSAIDS; antipyretic narcotics; antipyretic antidepressants, antipyretic selective serotonin reuptake inhibitors, antipyretic selective noradrenaline reuptake inhibitors, and antipyretic serotonin and norepinephrine uptake inhibitors.

60. The composition of claim 58, wherein the second antipyretic agent is selected from clonidine, venalfaxine, paroxetine, gabapentin, cycloheximide, dexamethasone, melanocortins, and nitric oxide.

61. The composition of claim 58, wherein the second antipyretic agent is an antipyretic non-steroidal anti-inflammatory agent (NSAID).

62. The composition of claim 61, wherein the antipyretic NSAID is selected from ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

63. The composition of claim 58, wherein the second antipyretic agent is an antipyretic steroidal anti-inflammatory agent.

64. The composition of claim 63, wherein the antipyretic steroidal anti-inflammatory agent is selected from cortodoxone, fludroracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone cyclopentylpropionate, flucetonide, fludrocortisone acetate, betamethasone benzoate, chloroprednisone acetate, clocortolone acetate, descinolone acetonide, desoximetasone, dichlorisone acetate, difluprednate, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

65. The composition of claim 58, wherein the second antipyretic agent is a natural product.

66. The composition of claim 65, wherein the natural product is selected from vertebrate organ, tissue, cell, protein and peptide products, vitamins, and plant products.

67. The composition of claim 66, wherein the plant product is selected from soy, black cohosh, chaste tree berry, dong quai, ginseng, evening primrose oil, motherwort, red clover, and licorice.

68. A composition for treating hyperthermia and eliciting analgesia in a mammalian subject comprising an antipyretic, analgesic effective amount of bicifadine or a pharmaceutically-acceptable salt, solvate, polymorph or prodrug thereof.

69. A composition for treating hyperthermia and eliciting analgesia in a mammalian subject comprising bicifadine and a non-bicifadine analgesic agent.

70. The composition of claim 69, wherein the non-bicifadine analgesic agent is selected from analgesic NSAIDs, analgesic narcotics, acetominophen, ibuprofen, ketoprofen, flurbiprofen, fenoprofen, loxoprofen, suprofen, aluminoprofen, pranoprofen, piroxicam, pentazocine, aspirin, acetanilide, phenacetin, diclofenac, antipyrine, aminopyrine, phenyl salicylate, methyl salicylate, methenamine, carprofen, choline salicylate, salsalate, diflunisal, dihydroergotamine mesylate, ergotamine tartrate, indomethacin, meclofenamate, mefenamic acid, naproxen, oxyphenbutazone, phenylbutazone, sulindac, tolmetin, and/or cyclooxygenase 2 (COX2) inhibitors.

71. The composition of claim 57, wherein said antipyretic effective amount of bicifadine is effective to prevent or alleviate peri-monopausal, menopausal, or post menopausal hot flashes in a mammalian female subject.

72. The composition of claim 57, further comprising a second therapeutic agent effective for treating one or more menopause-related symptom(s) or condition(s) in a mammalian female subject selected from hot flashes, depression, headache, palpitations, joint pain, loss of concentration, sleep disturbance, profuse perspiration, nervousness and irritability.

73. The composition of claim 72, wherein the second therapeutic agent is an antidepressant.

74. The composition of claim 73, wherein the anti-depressant is selected from monoamine oxidase inhibitors, selective serotonin reuptake inhibitors, tricyclic antidepressants, tetracyclic antidepressants, norepinephrine uptake inhibitors, selective noradrenaline reuptake inhibitors, and serotonin uptake inhibitors, and norepinephrine uptake inhibitors.

75. The composition of claim 73, wherein the anti-depressant is selected from venlafaxine, paroxetine, and citalopram.

76. The composition of claim 72, wherein the second therapeutic agent is an anxiolytic agent.

77. The composition of claim 57, wherein said antipyretic effective amount comprises between about 20 mg to about 1,200 mg of bicifadine.

78. The composition of claim 77, wherein said antipyretic effective amount comprises between about 25 mg to about 500 mg of bicifadine.

79. The composition of claim 78, wherein said antipyretic effective amount comprises between about 50 mg to about 250 mg of bicifadine.

80. The composition of claim 57, wherein said antipyretic effective amount of bicifadine is effective to lower an elevated temperature in said subject by about 0.5 5° F to about 5° F.

81. The composition of claim 57, wherein said antipyretic effective amount of bicifadine is effective to lower an elevated temperature in said subject by about at least 1 to 2 ° F.

82. A composition for treating hyperthermia and depression in a mammalian subject comprising an effective amount of bicifadine or a pharmaceutically-acceptable salt, solvate, polymorph or prodrug thereof, and an anti-depressant.

83. The composition of claim 82, wherein the anti-depressant is selected from monoamine oxidase inhibitors, selective serotonin reuptake inhibitors, tricyclic antidepressants, tetracyclic antidepressants, norepinephrine uptake inhibitors, selective noradrenaline reuptake inhibitors, serotonin uptake inhibitors, and norepinephrine uptake inhibitors.

84. The composition of claim 82, wherein the anti-depressant is venlafaxine, citalopram, or paroxetine.

85. A composition for treating one or more symptoms of peri menopause, menopause or post menopause in a mammalian female subject comprising an antipyretic effective amount of bicifadine.

86. The composition of claim 85, wherein said effective amount of bicifadine is an amount sufficient to prevent or alleviate one or more symptoms of peri menopause, menopause or post menopause selected from hot flashes, depression, headache, palpitations, joint pain, loss of concentration, cognitive dysfunction, mood disturbance, sleep disturbance, profuse perspiration, night sweats, palpitations, nervousness and/or irritability hot flashes, sleep disturbance, mood disturbance, cognitive dysfunction, depression, night sweats, palpitations, and anxiety.

87. The composition of 85, wherein said effective amount of bicifadine is an amount sufficient to prevent or reduce menopausal, post-monopausal, or peri menopausal hot flashes in said subject.

88. The composition of claim 85, further comprising one or more secondary therapeutic agents in an amount sufficient to prevent or alleviate one or more symptoms of peri menopause, menopause or post menopause selected from hot flashes, depression, headache, palpitations, joint pain, loss of concentration, cognitive dysfunction, mood disturbance, sleep disturbance, profuse perspiration, night sweats, palpitations, nervousness and/or irritability hot flashes, sleep disturbance, mood disturbance, cognitive dysfunction, depression, night sweats, palpitations, and anxiety.

89. The composition of claim 88, further comprising an antidepressant.
